(19) United States
(12) Patent Application Publication
(10) Pub. No.: US 2005/0112682 A9
(43) Pub. Date: May 26, 2005

(54) Two coloured fluorimetric protease assay

(76) Inventors: Rene Kuhlemann, (US); Andre Koltermann, Köln (DE); Ulrich Kettling, Köln (DE); Petra Schwille, Göttingen (DE)

Correspondence Address: David J Kulik;Wiley Rein & Fielding, Patent Administration, 1776 K Street N W, Washington, DC, 20006, US

(21) Appl. No.: 10/343,977

(22) PCT Filed: Aug. 7, 2001
(86) PCT No.: PCT/EP01/09112

(30) Foreign Application Priority Data
Aug. 7, 2000 (DE) 10038382.3

(51) Int. Cl. C12Q 1/70; G01N 33/53; C07H 21/04; C12P 21/04; C12P 21/02; C12N 5/06; C07K 14/435

(52) U.S. Cl. 435/7.1; 435/5; 435/69.7; 435/320.1; 435/325; 530/350; 536/23.5

(57) ABSTRACT

The invention concerns an autofluorescent fusion protein suitable for use as a protease substrate, a nucleic acid sequence which encodes this fusion protein, and a method of using the fusion protein and/or the nucleic acid sequence in a dual color, confocal fluorometric assay for the detection and quantification of proteolytic activity or proteolytic inhibitory activity in samples or cells.

The invention described herein concerns an autofluorescent fusion protein, which is suitable for use as a protease substrate, a nucleic acid sequence which encodes this fusion protein and a method using the fusion protein and/or the nucleic acid sequence in a dual-colour, confocal fluorometric assay for the detection and quantification of proteolytic activity in liquid samples or cells.

### BACKGROUND OF THE INVENTION

Proteases are enzymes which catalyze the hydrolytic cleavage of peptide molecules. The detection and quantitative determination of proteolytic activity is of significance for various research fields as well as for the pharmaceutical and biotechnical industries. Applicable test methods, known as protease assays, are used in the search for new enzymes with proteolytic activity, for their biochemical characterization, for the contamination control in production equipment and in the search for substances with activity modifying properties. Particular focus here is on high-throughput screening for effective substances with protease-inhibiting effects (protease inhibitors), e.g., for use in the treatment of viral infections.

Primarily peptides are used as substrates in protease assays. Their amino acid sequences permit ideal interaction with the active centre of the proteases. A series of suitable methods for measuring the hydrolysis of these substrates is described in the literature, e.g., electrophoretic, chromatographic or spectroscopic methods (Methods Enzymol. (1981) 80:341-361, Methods Enzymol. (1994) 241:70-86, Peptides (1991) 787-789). The known methods for the analysis of protease activities can basically be categorized either as heterogeneous or homogeneous methods. In the case of heterogeneous methods, the analysis of the hydrolysis products is performed separated from the reaction (off-line); methods such as SDS-PAGE, Western Blot, ELISA or HPLC are used here (J. Immunol. Methods (1993) 161:151-155). Besides the disadvantage of the chronological disparity between reaction and determination of the measurement values, these methods are characterized especially by the complex handling. Their advantage lies in the possibility of being able to use completely unmodified substrates. Heterogeneous methods are therefore primarily used for single analyses and evaluations.

The advantage of homogeneous analysis methods is that the reaction can be observed directly in real time (on-line). Yet another advantage of spectroscopic methods in comparison with other methods is that only a relatively small amount of the substance is required for the measurement. The homogeneous methods of the first generation included spectroscopic methods using chromogenic or fluorogenic substrates. The measurement in these cases is based on the change in the absorption spectrum of a chromophore (chromogenic substrate) or the fluorescence emission of a fluorophore (fluorogenic substrate) as a result of the proteolytic cleavage (Biochemistry (1967) 67(6):1765-1777, Anal. Biochem. (1979) 99(1):53-64). Fluorogenic substrates generally have the advantage of higher sensitivity in comparison with chromogenic substrates so that substantially lower concentrations can be detected. Chromophores and fluorophores in these substrates are usually C-terminal coupled via an amide bond to a peptide with an amino acid sequence specific for the protease to be determined.

However, these substrates of the first generation suffer from a number of disadvantages which limit their use. To begin with, these chromophores and fluorophores are, for the most part, comparatively large, aromatic residues (e.g., p-nitrophenol, β-naphtylamide, etc.) which differ strongly in their chemical nature from the residues of the twenty amino acids which occur naturally. Moreover, the amide bond that is normally used for the coupling of the chromophore or the fluorophore differs substantially from a peptide bond. This drastically reduces the enzyme's selectivity and the transformation rate.

Finally, the chromophore or the fluorophore must usually be bonded directly C-terminal to the protease cleavage site so that it can be released during the hydrolysis catalyzed by the protease and so change its spectral or fluorescent properties.

These substrates are completely unsuitable for the analysis of substrate specificities on the peptide chain at some distance from the protease cleavage site, i.e., the secondary substrate specificity. For example, the protease from HIV (human immunodeficiency virus) requires three or four amino acid residues on each side of the cleavage site for the substrate recognition. These types of assays require longer peptides and the possibility of varying the sequence within these peptide chain. Fluorescent protease substrates which display an intramolecular energy transfer have been introduced for this purpose (Castillo, M. J. et al., Anal. Biochem. 95 (1979), 228-235; Gershkovich, A. A., and Kholodovych, V. V., J. Biochem. Biophys. Methods 33 (1996), 135-162). One method is to attach a fluorophore (donor) at one end of the substrate peptide and a quencher molecule (acceptor, e.g., dabcyl) at the other end (Matayoshi, E. D. et al., Science 247 (1990), 954-958; Methods in Enzymology (1994) 241:70-86; Anal. Biochem. (1995) 227:148-155). In the intact peptide substrate, the quencher extinguishes the fluorescence of the fluorophore almost completely. But the cleavage reaction separates the quencher and the fluorophore, causing a strong rise in the measurable fluorescence emission resulting from the excitation of the fluorophore. A second fluorophore whose excitation spectrum overlaps with the emission spectrum of the first one can also take the place of the quencher molecule, leading to a fluorescence resonance energy transfer (FRET, Förster energy transfer). During methods which are based on these substrates, the quality of the signal and consequently the sensitivity of the assay are especially dependent on the distance between the donor and the acceptor, on the spectral overlap between the emission of the donor and the absorption of the acceptor and on the orientation of the transition dipoles. Consequently, these three parameters restrict the applicability of such protease substrates. The minimum length of seven amino acid residues required for the HIV protease assay mentioned above represents a considerable restriction of the assay. A corresponding substrate therefore requires the careful selection of donor and acceptor molecule and a synthesis which takes the steric criteria into account (Wang, G. T. et al., Tetrahedron Letters 31 (1990), 6493).

Alternatively to such protease assays based on the measurement of a fluorescence energy transfer between two chemical groups, the literature knows of peptide substrates for application in the dual-colour, confocal fluorometry (WO 99/34195; Koltermann, A. et al., in: Fluorescence Correlation Spectroscopy—Theory and Application, Springer-Verlag, Rigler, R., Elson, E. (eds.), (2000)). In these substrates, a fluorophore is chemically linked to both ends of the peptide chain, whereby the two fluorophores display spectrally divergent fluorescence emission properties. By using confocal fluorometric methods such as the dual-colour fluorescence cross-correlation spectroscopy (dual-colour FCS, DE 197 57 740) or the confocal fluorescence coincidence analysis (CFCA, WO 99/34195), it is possible to determine the proportion of molecules to which both fluorophores are linked in a test solution. As the only condition for this is the linking of the fluorophores by means of a chemical bond, the determination of the proteolytic activity without being restricted by the boundary conditions described above is possible: the distance and the orientation between the fluorophores is freely selectable, depending on the assay requirements. In contrast to the methods mentioned before, this assay principle makes it possible to follow protease assays on-line under virtually natural conditions as entire protein domains can be used as cleavage site and the spatial orientation of the two fluorophores to each other is irrelevant. In addition, with this method sub-nanomolar concentrations of dual-colour fluorophores can be detected even in the smallest sample volumes (of only a few picolitres).

However, a disadvantage of such known protease substrates with chemically linked fluorophores is the complex method required to synthesize them. Although peptides can be synthesized relatively efficiently by solid-phase synthesis up to a length of about 50 amino acids, the double, site-specific coupling of fluorophores requires considerable effort, as the fluorophores used are usually not compatible with the peptide synthesis chemistry. And the same substantial effort is required to synthesize even slightly modified peptide sequences, such as for the analysis of a slightly modified substrate specificity. It is considerably simpler to produce polypeptides using genetic engineering methods, i.e., by constructing a nucleic acid sequence which allows the expression of a polypeptide coded by the nucleic acid sequence using cellular.or cell-free expression systems.

Furthermore, none of the substrates known to date does meet the requirements of an intracellular protease assay. Many proteases are not secreted by the cells into the medium, but are intracellular enzymes; in the case of eukaryotic cells, they appear specifically only in single compartments. Disruption of the cells with the goal of making the proteases accessible to measurement is usually a complex process. In addition, the spatial resolution is lost.

On the other hand, it is precisely the intracellular fluorescence measurement with spatial resolution which is made possible by modern confocal fluorescence methods (Schwille, P. et al., Biophys. J. 77 (1999), 2251). To be able to measure protease activities intracellularly by fluorometric methods, the appropriate substrate must be inserted into the cell or produced there. Methods for inserting substances are known, e.g., by micro-injection or electroporation. These methods, however, all have the problem that they are relatively labor-intensive, that they principally damage the cells and that a potential control of quantity and site of the insertion is difficult. Alternatively, however, a nucleic acid sequence encoding the protease substrate can be inserted into the cell using known molecular biological methods. The subsequent expression of the nucleic acid sequence into the corresponding polypeptide, which can also be controlled externally, results in the protease substrate. Such protease substrates are then accessible for a fluorescence measurement if the code for one or more autofluorescent proteins (AFPs) is linked to the code for the original protease substrate.

The possibility of such a completely expressible protease substrate is known in the literature (Mitra, R. D. et al., Gene 173 (1996), 13-93). This purpose is served by a nucleic acid sequence which contains the code for a protease cleavage site between sequences coding for two variants of GFP (Green Fluorescent Protein, from Aequorea Victoria). The GFP variants are selected in such a way that a fluorescence-energy transfer between them is measurable for an intact peptide bridge (a bridge with 20 amino acid residues and a cleavage site for Factor Xₐ is described). Proteolytic activity leads to a separation of the two fluorophores and thus to a decrease in the fluorescence emission of the acceptor fluorophore. This method solves the problems involved with substrates which are chemically synthesized and is, basically , suitable for intracellular assays. Nevertheless, it is subject to all of the disadvantages of fluorescence energy transfer substrates described above, such as the restriction in the structural design of the substrate and the maximum possible length of the protease recognition sequence.

The technical problem underlying the present invention is to design a protease substrate and to provide a method for the measurement of proteolytic activity using this protease substrate which, when taken together, would avoid the disadvantages described above of known protease substrates and known methods for the measurement of proteolytic activity. In particular, the substrate should be suitable for synthesis by means of cellular or cell-free expression systems and enable the intracellular analysis of protease activities. It should avoid the necessity of the complex, region-specific chemical coupling of fluorophores to polypeptides. Finally, it should allow the unrestricted design of the protease cleavage site.

### SUMMARY OF THE INVENTION

Surprisingly, it was found that a special autofluorescent fusion protein, which consists of two distinguishable autofluorescent proteins accomplishes the requirements described above. The invention therefore pertains to
(1) an autofluorescent fusion protein which consists of a first autofluorescent protein, a cleavage site segment with a protease cleavage site and at least one further autofluorescent protein distinguishable from the first autofluorescent protein, whereby there is no significant fluorescence energy transfer between the two autofluorescent proteins;
(2) a nucleic acid sequence which is coding for an autofluorescent fusion protein as defined in (1);
(3) a vector, comprising a nucleic acid sequence as defined in (2);
(4) a cell or a transgenic organism, comprising the nucleic acid sequence as defined in (2) and/or the vector as defined in (3);
(5) a method for production of the autofluorescent fusion proteins as defined in (1), comprising the expression of the nucleic acid sequence as defined in (2) with the help of a cellular or cell-free expression system;
(6) a method for analysis of a sample for proteolytic activity, comprising the steps:
   (a) combining of the autofluorescent fusion protein as defined in (1) with the sample to be tested for proteolytic activity in an aqueous test solution;
   (b) incubation. under conditions which are suitable for proteolytic cleavage; and
   (c) measurement of the proportion of split fusion protein by means of confocal fluorometric methods;
(7) a method for analysis of a sample for protease inhibiting activity, comprising the steps:
   (a) combining of the autofluorescent fusion protein as defined in (1) with the sample to be tested for protease inhibiting activity and the appropriate protease in an aqueous test solution;
   (b) incubation under conditions which are suitable for proteolytic cleavage; and
   (c) measurement of the proportion of split fusion protein by means of confocal fluorometric methods;
(8) a method for analysis of intracellular protease activity, comprising the steps:
   (a) insertion of the nucleic acid sequence as defined in (2) and/or the vector as defined in (3) into the celI to be tested so that the autofluorescent fusion protein as defined in (1) is expressed intracellularly;
   (b) incubation under conditions which are suitable for an expression and proteolytic cleavage of the fusion protein; and
   (c) determination of the protease activity occurring intracellularly by means of confocal fluorometric methods; and
(9) a method for analysis of intracellular protease inhibiting activity, comprising the steps:
   (a) insertion of the nucleic acid sequence as defined in (2) and/or the vector as defined in (3) into the cell to be tested so that the autofluorescent fusion protein as defined in (1) is expressed intracellularly;
   (b) incubation under conditions which are suitable for an expression and proteolytic cleavage of the fusion protein; and
   (c) determination of the protease inhibiting activity occurring intracellularly by means of confocal fluorometric methods.

The invention will be described in detail as follows.

### DESCRIPTION OF FIGURES

FIG. 1: Schematic exemplary depiction of the inventive fusion protein B (cf. also SEQ ID NO:2). The first autofluorescent protein (AFP) is rsGFP, the second DsRed, between the two linker sequences a specific cleavage site for the protease from the tobacco etch virus (TEV) is inserted.

FIG. 2: Fluorescence emission spectra of three fusion protein variants with the excitation wavelength 488 nm; AFP 1 is rsGFP, AFP 2 is DsRed. In variant 1 (fusion protein A; SEQ ID NO:1) there are 13 amino acids, in variant 2 (fusion protein B; SEQ ID NO:2) 32 amino acids, in variant 3 (fusion protein C; SEQ ID NO:3) 73 amino acids between the two autofluorescent proteins. The decrease of the FRET related to the increasing length of the linkers can clearly be recognized by the decreasing emission of the DsRed. The inventive use of the fusion proteins as protease substrates and their measurements by means of cross-correlation analysis or coincidence analysis remain unaffected by this.

FIG. 3: Measurement of the two-photon cross-correlation during the cleavage of the fusion protein in variant 2 (fusion protein B; SEQ ID NO:2) as a consequence of the effect of the TEV protease. The cleavage of the bond and the consequent reduction of the proportion of double fluorescing molecules in the measurement solution cause the decrease of the cross-correlation amplitude.

FIG. 4: Measurement of the two-photon auto-correlation curves during the cleavage of the fusion protein in variant 2 (fusion protein B; SEQ ID NO:2) effected by the TEV protease. The cleavage of the bond does neither measurable affect the fluorescence intensity nor the concentration or particle size of the two AFPs.

FIG. 5: Chronological progress of the two-photon cross-correlation amplitude during the cleavage of the fusion protein in variant 2 (fusion protein B; SEQ ID NO:2) for varying quantities of the TEV protease.

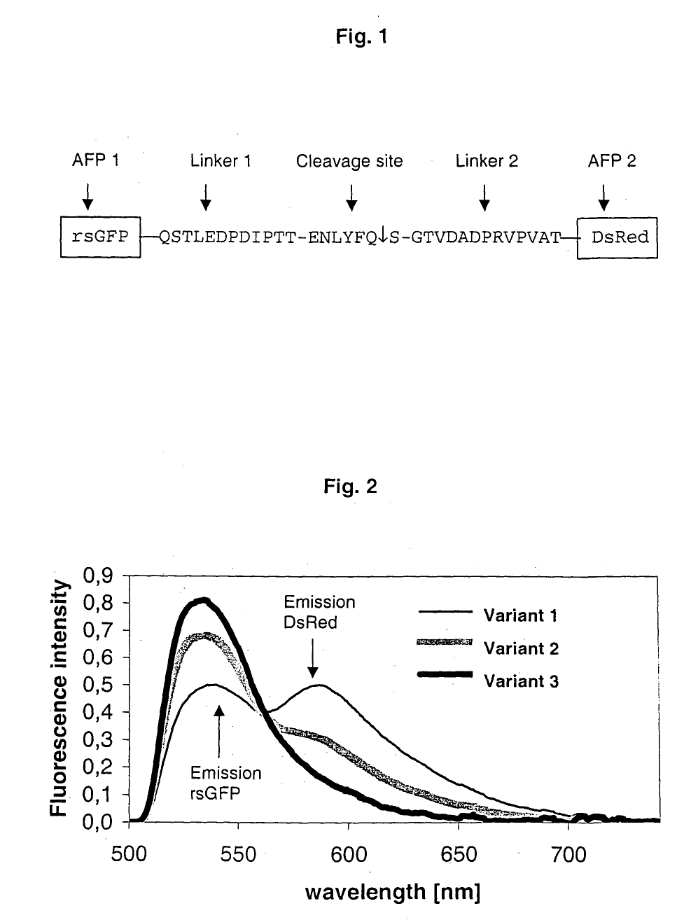

### DETAILED DESCRIPTION OF THE INVENTION

The fusion protein of embodiment (1) of the invention consists of several partial sequences which together form a continuous protein strand. According to the invention, the protein strand contains a first autofluorescent protein, a cleavage site segment with a protease cleavage site and at least one further autofluorescent protein distinguishable from the first autofluorescent protein whereby there is no significant fluorescence energy transfer between the two autofluorescent proteins. In a preferred embodiment, the cleavage site segment in the protein strand is located between the two autofluorescent polypeptides which can be distinguished from one another on the basis of their spectral properties.

“No significant fluorescence energy transfer” in the sense of the present invention means that the emitted fluorescence of the two (or more) autofluorescent proteins does not excite the other autofluorescent protein, or excites it by less than 50%, preferably by less than 30%, most preferably by less than 10%. This can, on the one hand, be influenced by the distance between the two fluorescence proteins, i.e., by the length of the spacer peptide between the autofluorescent proteins (here cleavage site segment). On the other hand, the type of the spacer protein, i.e., the degree to which the spacer peptide tends to form secondary structures (wrinkles), is relevant for the fluorescence energy transfer. In the sense of the present invention, particular preference is given to cleavage site segments (with protease cleavage site) which do not form secondary structures and are relatively rigid. The cleavage site segments of the present invention have particularly a length of at least 10, preferably at least 20, and most preferably at least 30 amino acid residues.

According to this invention, the autofluorescent proteins are proteins which display fluorescent properties following expression by means of a cellular protein biosynthesis or by means of a cell-free system for ribosomal protein synthesis and, optionally after subsequent modification by cellular components or added enzymes.

“Spectrally distinguishable” in the sense of the present invention are fluorophores used in the invention in particular when- their emission spectra can be distinguished from each other. In a preferred embodiment, in the one hand the green fluorescent protein from Aequorea victoria or a variant, particularly a red-shifted variant, and on the other hand the DsRed from Discosoma sp. or a variant thereof are used as autofluoresent proteins. Particularly preferred is the use of a fusion protein with rsGFP (cf. SEQ ID NO:1, aa 11 to 249) and DsRed (SEQ ID NO:1, aa 263 to 487).

Particularly suitable as protease cleavage sites are those which are specifically recognized and split by the protease from HIV (human immunodeficiency virus; recognizes the aa sequence SQNYPIVQ), by the protease from the Hepatitis C virus, by the protease from TEV (tobacco etch virus; recognizes the aa sequence ENLYFQS), by the protease from hCMV (human cytomegalovirus; recognizes the aa sequence RGVVNASSRLA), by the protease from HSV (herpes simplex virus; recognizes the aa sequence LVLASSSF), by the protease plasmin (recognizes the aa sequence KXYK), by the protease ACE (angiotensin converting enzyme; recognizes the aa sequence GKYAPWV), by the protease tPA, by Factor Xₐ (recognizes the aa sequence IEGR), thrombin (recognizes the aa sequence VGPRSFLLK), etc.

Particularly preferred fusion proteins are the following fusion proteins A-C (the cleavage site segment is underlined, the protease cleavage site is shown in bold; ↓ indicates the cleavage site for the TEV protease).

Fusion protein A (variant 1; 487 ^{aa}; SEQ ID NO:1):
MTMITPSLHAMASKGEELFTGVVPILVELDGDVNGHKFSVSGEGEGDATYGKLTLKFICTT
GKLPVPWPTLVTTLCYGVQCFSRYPDHMKRHDFFKSAMPEGYVQERTIFFKDDGNYKTRAE
VKFEGDTLVNRIELKGIDFKEDGNILGHKLEYNYNSHNVYIMADKQKNGIKVNFKTRHNIE
DGSVQLADHYQQNTPIGDGPVLLPDNHYLSTQSALSKDPNEKRDHMVLLEFVTAAGITHGM
DELYNQSTLEDPRVPVATMRSSKNVIKEFMRFKVRMEGTVNGHEFEIEGEGEGRPYEGHNT
VKLKVTKGGPLPFAWDILSPQFQYGSKVYVKHPADIPDYKKLSFPEGFKWERVMNFEDGGV
VTVTQDSSLQDGCFIYKVKFIGVNFPSDGPVMQKKTMGWEASTERLYPRDGVLKGEIHKAL
KLKDGGHYLVEFKSIYMAKKPVQLPGYYYVDSKLDITSHNEDYTIVEQYERTEGRHHLFL
Fusion protein B (variant 2; 506 ^{aa}; SEQ ID NO:2):
MTMITPSLHAMASKGEELFTGVVPILVELDGDVNGHKFSVSGEGEGDATYGKLTLKFICTT
GKLPVPWPTLVTTLCYGVQCFSRYPDHMKRHDFFKSAMPEGYVQERTIFFKDDGNYKTRAE
VKFEGDTLVNRIELKGIDFKEDGNILGHKLEYNYNSHNVYIMADKQKNGIKVNFKTRHNIE
DGSVQLADHYQQNTPIGDGPVLLPDNHYLSTQSALSKDPNEKRDHMVLLEFVTAAGITHGM
DELYNQSTLEDPDIPTTENLYFQ↓SGTVDADPRVPVATMRSSKNVIKEFMRFKVRMEGTVNG
HEFEIEGEGEGRPYEGHNTVKLKVTKGGPLPFAWDILSPQFQYGSKVYVKHPADIPDYKKL
SFPEGFKWERVMNFEDGGVVTVTQDSSLQDGCFIYKVKFIGVNFPSDGPVMQKKTMGWEAS
TERLYPRDGVLKGEIHKALKLKDGGHYLVEFKSIYMAKKPVQLPGYYYVDSKLDITSHNED
YTIVEQYERTEGRHHLFL
Fusion protein C (variante 3; 547 ^{aa}; SEQ ID NO:3):
MTMITPSLHAMASKGEELFTGVVPILVELDGDVNGHKFSVSGEGEGDATYGKLTLKFICTT
GKLPVPWPTLVTTLCYGVQCFSRYPDHMKRHDFFKSAMPEGYVQERTIFFKDDGNYKTRAE
VKFEGDTLVNRIELKGIDFKEDGNILGHKLEYNYNSHNVYIMADKQKNGIKVNFKTRHNIE
DGSVQLADHYQQNTPIGDGPVLLPDNHYLSTQSALSKDPNEKRDHMVLLEFVTAAGITHGM
DELYNQSTLEDPTSQHGTNSEIEEYLKVLYDYDIPTTENLYFQ↓SGTVDAGADAGKKKDQK
DDKVAEQASKDPRVPVATMRSSKNVIKEFMRFKVRMEGTVNGHEFEIEGEGEGRPYEGHNTV
KLKVTKGGPLPFAWDILSPQFQYGSKVYVKHPADIPDYKKLSFPEGFKWERVMNFEDGGVV
TVTQDSSLQDGCFIYKVKFIGVNFPSDGPVMQKKTMGWEASTERLYPRDGVLKGEIHKALK
LKDGGHYLVEFKSIYMAKKPVQLPGYYYVDSKLDITSHNEDYTIVEQYERTEGRHHLFL

The method according to embodiment (6) of the invention is a method for the analysis of a sample for proteolytic activity, comprising the steps:
(1) Combining of the inventive autofluorescent fusion protein produced by a cellular or cell-free expression system with the sample to be examined for proteolytic activity in an aqueous test solution;
(b) Incubation under conditions which are suitable for proteolytic cleavage;
(c) Measurement of the proportion of split fusion protein by means of confocal fluorometric methods.

As used in the invention, a sample being tested can be a natural isolate, a chemical component from a substance library, a fraction from a fermentation broth or a protease library created by means of variation techniques.

Confocal fluorometric methods are methods which evaluate fluorescent signals based on the passage of single fluorophores through an excitation focus. Confocal fluorometric methods include in particular fluorescence correlation spectroscopy (FCS), dual-colour fluorescence cross-correlation spectroscopy (KK-FCS), confocal fluorescence coincidence analysis (CFCA) and the 2D fluorescence intensity distribution analysis (2D-FIDA).

The method according to embodiment (7) of the invention is a method for the analysis of a sample for protease inhibiting activity. This method is analogous to the method described above in embodiment (6), whereby a sample to be tested for protease inhibiting activity, together with the appropriate protease, is used in place of the sample to be tested for proteolytic activity.

As used in the invention, the appropriate protease is to be understood as one of the proteases which have a sequence specificity congruent with the protease substrate.

According to embodiment (8) the invention pertains to a method for the analysis of intracellular protease activity. This method is analogous to the method described above in form (6), whereby a nucleic acid sequence coding for the fusion protein is inserted into cells in such a way that it is expressed intracellularly, and the intracellularly occurring protease activity can be analyzed by means of.the confocal fluorometric measurement.

According to embodiment (9), the invention pertains to a method for the analysis of intracellular protease inhibiting activity analogous to the methods (6) to (8) described above, whereby the nucleic acid sequences coding for the fusion protein and for the appropriate protease are inserted into the cells in such a manner that they are expressed intracellularly, and the intracellularly occurring protease inhibiting activity can be analyzed by means of the confocal fluorometric measurement.

In a preferred embodiment, the insertion of the nucleic acid sequence in eukaryotic cells is performed by using a vector which allows a locally and temporally controlled expression. In a particularly preferred embodiment, the expression occurs only in selected cell compartments.

In another particularly preferred embodiment, the dual-colour fluorescence cross-correlation spectroscopy (KK-FCS), the confocal fluorescence coincidence analysis (CFCA) or the 2D fluorescence intensity distribution analysis (2D-FIDA) is used as the confocal fluorometric measurement method.

According to the invention, the proportion of molecules displaying a fluorescence energy transfer can be determined as a further measurement parameter in addition to the dual-colour confocal fluorometric measurement methods. In a further, particularly preferred embodiment, the method for the screening-based, evolutive optimization of biomolecules with proteolytic activity or the screening-based, evolutive generation of biomolecules with proteolytic activity is used.

The invention will be more closely described on the basis of the following non-exhaustive example.

### EXAMPLE

Construction and Purification of a Fusion Protein Made from rsGFP and Its Use in an Assay for the TEV Protease

Strategy of cloning: As minimal quantities of the substance are adequate for the performance of FCS measurements, a vector with lac promoter was used for expression of the fusion proteins, whereby the risk of the formation of inclusion bodies in the bacteria (inclusion bodies) was minimized. To enable an alternative purification with the aid of nickel chelate chromatography, a C-terminal hexahistidine sequence was attached to all of the fusion proteins intended for the expression in E. coli. As the extent to which the amino acid residues in the environment of the protease recognition sequence would affect the reaction was not known, a peptide sequence obtained from the polyprotein of the tobacco etch virus was selected for insertion between the two fluorophores. This sequence is in the region of the cleavage site between capsid protein and polymerase of the virus. The accessibility of the cleavage site for the TEV protease depends, among other factors, on the length of the peptide sequence between the two autofluorescent protein parts. For this reason, a short variant with an cleavage site segment of 32 amino acid residues (variant 2; STEV; SEQ ID NO:2) and a long variant with 73 amino acid residues (variant 3; LTEV; SEQ ID NO:3) was produced, with the cleavage site being in the middle in each case. The amino acids from 2761 to 2819 were therefore selected from the TEV polyprotein for the long insert, the amino acids from 2781 to 2797 for the short insert.

As a first step, two constructs were produced, in each of which the gene for rsGFP was inserted before the gene for DsRed and located in the same reading frame with the latter. For the expression in E. coli, first the GFP gene of the vector pQBI63 (Qbiogene) was amplified . The PCR product was digested with the restriction endonucleases Sal I and Sph I and the insert then inserted into the plasmid pDsRed (Clontech) which had been cut the same way. The result of the cloning was controlled by digestion of the plasmids with the restriction enzymes previously used. The final sequencing showed that the cloning had been performed successfully and without mutations (“pGFRed”; variant 1; SEQ ID NO:1). In the next step, the gene for the fusion protein was extended at its 3′ end by a nucleotide sequence coding for six histidine residues following each other in a series in order to enable a later purification of the proteins by nickel chelate chromatography.

This was done analogously to the method described above by amplification of the DsRed gene of the plasmid pDsRed. The insert and the plasmid pGFRed were then cut with the restriction enzymes Sal I and Not I. The construct obtained following the ligation is designated pGFRed-CH in the following. The result of the cloning was also tested here by digestion of the plasmid DNA with the restriction enzymes used and by sequencing.

The constructs described above (pGFRed, pGFRed-CH) possess a short nucleotide sequence from the remaining residue of the 5′-MCS between the genes of the two fluorescent proteins. These sections were expanded in the following by the insertion of synthetically produced sequences. In this way the length of the peptide chain between the two fluorescent proteins could be varied and a specific protease cleavage site could be inserted in the protein later formed.

Two peptide sequences of different lengths were designed with a cleavage site for the TEV protease unique to the entire protein in the middle; and which were integrated into both the original GFRed protein and into the GFRed protein containing the polyhistidine sequence. The sequences were generated by the direct insertion of a synthetic nucleotide sequence. For this purpose, the vectors were cut with BamH I and the phosphate groups of the free ends were removed using alkaline phosphatase. The two synthetic oligonucleotides were phosphorylated so that they could be inserted into the vectors as double strands. The resulting plasmids are pSTev (variant 2; SEQ ID NO:2; short insert), pSTev-CH (short insert with C-terminal hexahistidine sequence), pLTev (variant 3; SEQ ID NO:3; long insert) or pLTV-CH (long insert with C-terminal hexahistidine sequence).

As only one cleavage site is used, the gene segments can be inserted in two different orientations. The orientation of the inserts was controlled by an analytic PCR. A PCR product resulted only if the gene segment had been inserted in the correct orientation. The method itself can also be used on other restriction cleavage sites. The type and position of the protease cleavage site within the later protein can also be freely selected by the appropriate selection of the inserted nucleotide sequence.

Protein expression in E. coli: Clones with the appropriate vector construct from a permanent culture were smeared on a selective nutrient agar plate and incubated overnight at 37° C. One colony was used for inoculation of a pre-culture of 10 ml selective medium. This was also shaken overnight at 37° C. and served the next morning as inoculum for two batches of 200 ml selective medium, each of which was inoculated with 5 ml pre-culture. When the cultures had reached an optical density of 0.7-0.8 at 600 nm, the protein expression was induced by the addition of 100 μl IPTG solution (0.5 mM final) to each. After four hours, the bacteria were centrifuged off in the refrigerated centrifuge at 4° C. and 5000 g for 15 minutes and the supernatant was discarded.

Disruption of the bacteria: The bacteria were resuspended in 20 ml buffer, then pressed with a syringe through a needle with a diameter of 0.25 mm, finally lysated in the French Press. The lysate was collected in a ice-cold centrifuge tube to which 100 μl of a solution of 100 mg/ml PMSF in ethanol for the inhibition of proteases had been added. The cell debris was immediately centrifuged off in the refrigerated centrifuge at 4° C. and 20000×g for 20 minutes. The bacteria lysate was pressed through a syringe filter and an aliquot of 100 μl was removed before application to the column.

Protein purification by anionic exchange chromatography: DEAE Sephacel was used as column material, the column diameter was 1 cm and the filling height 5 cm. The column was packed free of bubbles and equilibrated with a minimum of 50 ml wash buffer before loading; the flow rate was 5 ml/min. The bacteria were disrupted in the wash buffer and the columns were loaded with the filtered supernatant; the flow was collected for later analysis. The column was then washed with 40 ml buffer and the flow here was also collected. The protein bonded to the column material was eluated over a linear gradient of 50 mM to 1 M sodium chloride in wash buffer and fractions of 30 drops each (about 2 ml) were collected; the total volume was 100 ml. Finally, the column was regenerated with 2 M sodium chloride in wash buffer and subsequently equilibrated with 50 ml wash buffer. All of the fractions were tested for fluorescence at the excitation wavelengths of 488 nm and 543 nm. The fractions with strong fluorescence were pooled.

Protein purification by nickel chelate chromatography: A HiTrap chelating column (Pharmacia) with 1 ml column volume was used; the flow rate was 0.5 ml/min for the application of the lysate, and otherwise 1 ml/min. The column was first washed with 10 ml water and then loaded with 2 ml nickel chloride solution (100 mM). Unspecifically bound Ni²+ ions were removed by 10 ml of a solution of 300 mM imidazole in the wash buffer; the column was then equilibrated with 10 ml wash buffer. The filtered supernatant of the disrupted bacteria was applied to the column, which was subsequently washed with 10 ml buffer and the protein then eluated with 8 ml 100 mM imidazole in wash buffer. The collected fractions of 1 ml each were examined for fluorescence and the column washed with 10 ml 500 mM imidazole in buffer. The flow from the loading, washing and cleaning steps was collected and preserved for later analysis.

The samples to be tested were concentrated with the aid of a centricon column at 1000×g, mixed with 100 μl PBS buffer and centrifuged again at 1000×g. For the measurement, they were diluted with PBS buffer so that the measurement values would be in a reasonable range.

Fluorescence spectra: Emission spectra at an excitation wavelength of 488 nm were taken of each sample. FIG. 2 shows the emission (λ_{Ex.}=488 nm) spectra of the three fusion proteins GFRed, STev and LTev cleaned up using anionic exchange chromatography. The spectra have each been standardized for the totals of the fluorescence intensities at 540 nm and 580 nm so that the intensities relative to each other can be compared. These two wavelengths were selected for the norming as the maximums of DsRed and rsGFP are in this range; the sum of the two gives an approximate measurement for the total intensity.

It is striking that the graphs of the preparations GFRed and STev show, besides the expected maximum of the GFP emission at about 540 nm, a second maximum or a shoulder at about 580 nm; however, this is not to be seen in the spectrum of the protein LTev. This phenomenon was caused by energy transfer (FRET) within the protein. This energy transfer results from the overlapping of the emission spectrum of the rsGFP with the excitation spectrum of the DsRed. The proportion of the energy transfer falls as the linker length increases.

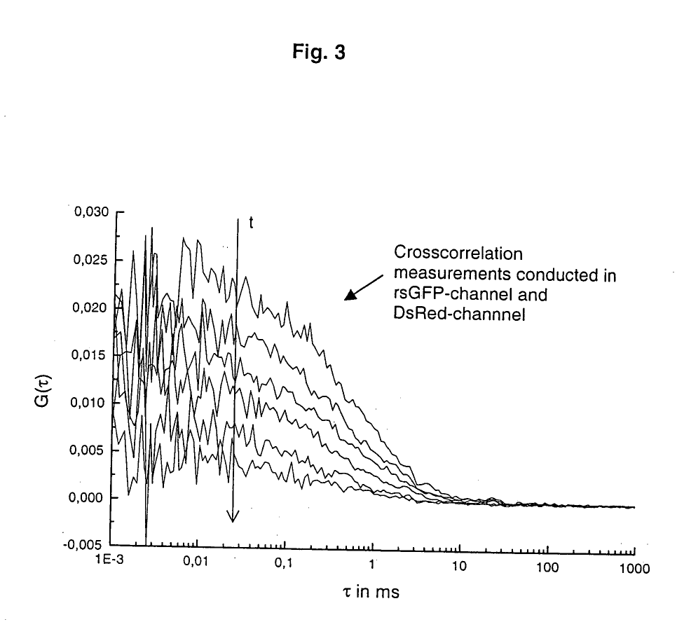

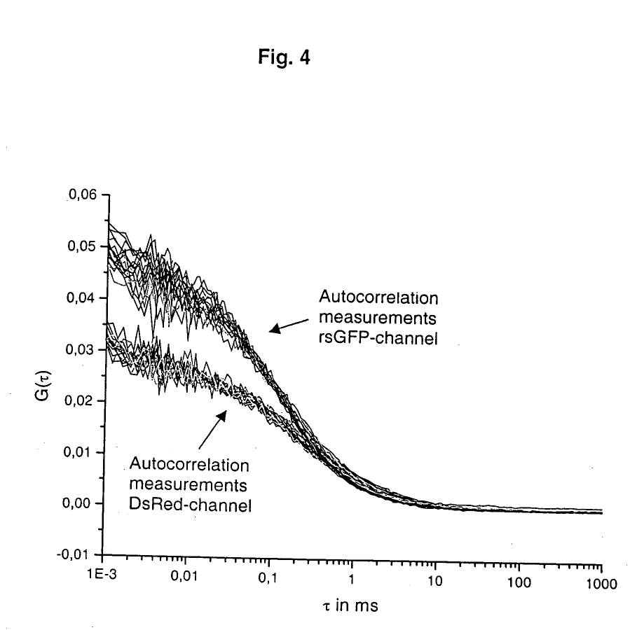

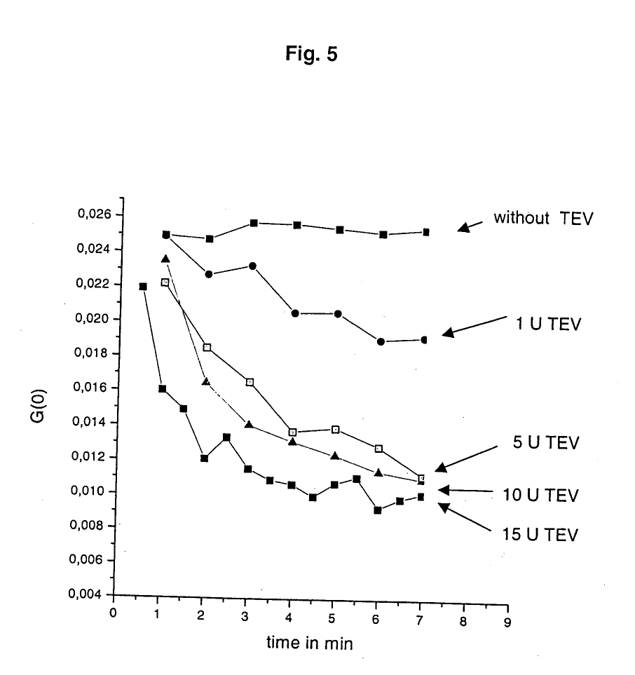

Fluorescence correlation spectroscopic measurements: The measurement of the proteolytic activity of the TEV protease with the substrate proteins prepared from E. coli was performed in a Nunc test chamber with a self-developed FCS device. The substrate concentration amounted to 50 nM. Two batches of each substrate consisting of 100 μl substrate in PBS buffer were prepared; one batch was mixed with the TEV protease (various concentrations) and the other served as a negative control. The measurement was conducted under two-photon excitation at a wavelength of 950 nm. Following colour separation, detection was performed via a dichroite (D530) in the two ranges 500 nm to 550 nm (rsGFP channel, detection filter: 525DF50) and 560 nm to 610 nm (DsRed channel, detection filter: 585DF50). The measurement time was 60 s; kinetics were observed online for several minutes. The measurements are shown in FIGS. 3 to 5.

1. An autofluorescent fusion protein which consists of a first autofluorescent protein, a cleavage site segment with a protease cleavage site and at least one further autofluorescent protein distinguishable from the first, whereby there is no significant fluorescence energy transfer between the two autofluorescent proteins.

2. Autofluorescent fusion protein according to claim 1, whereby the cleavage site segment
(i) is located between two autofluorescent polypeptides, distinguishable from one another by their spectral properties, and/or
(ii) includes terminal linker peptides in addition to the protease cleavage site, and/or
(iii) has a length of at least 10, preferably more than 20, most preferably at least 30 amino acid residues.

3. Autofluorescent fusion protein according to claim 1 or 2, wherein the cleavage site segment has a protease cleavage site which is recognized and split specifically by the protease from human immunodeficiency virus, by the protease from the hepatitis C virus, by the protease from the tobacco etch virus, by the protease from human cytomegalovirus, by the protease from herpes simplex virus, by the protease plasmin, by the protease angiotensin converting enzyme, by the protease tPA of Factor Xₐ and/or thrombin.

4. Autofluorescent fusion protein according to one of the claims 1 to 3, wherein the first autofluorescent protein is the green fluorescent protein from Aequorea Victoria (GFP) or a variant, particularly a red-shifted variant (rsGFP) thereof, and the second autofluorescent protein is the DsRed from Discosoma sp. or a variant thereof.

5. Autofluorescent fusion protein according to one of the claims 1 to 4, which further includes other functional peptide sequences, in particular signal peptides, affinity marker peptides or detection marker peptides.

6. Autofluorescent fusion protein according to claim 1 which includes the sequence of fusion protein B or C (SEQ. ID. NOs:2 or 3).

7. A nucleic acid sequence which is coding for an autofluorescent fusion protein as defined in claims 1 to 6.

8. A vector, comprising a nucleic acid sequence as defined in claim 7.

9. A cell or transgenic organism, comprising the nucleic acid sequence according to claim 7 and/or the vector according to claim 8.

10. A method for production of the autofluorescent fusion protein according to one of the claims 1 to 6, comprising the expression of the nucleic acid sequence according to claim 7 with the help of a cellular or cell-free expression system.

11. A method for analysis of a sample for proteolytic activity, comprising the steps:
(a) combining of the autofluorescent fusion protein as defined in claims 1 to 6 with the sample to be tested for proteolytic activity in an aqueous test solution;
(b) incubation under conditions which are suitable for proteolytic cleavage; and
(c) measurement of the proportion of split fusion protein by means of confocal fluorometric methods.

12. A method for analysis of a sample for protease inhibiting activity, comprising the steps:
(a) combining of the autofluorescent fusion protein as defined in the claims 1 to 6 with the sample to be tested for protease inhibiting activity and the appropriate protease in an aqueous test solution;
(b) incubation under conditions which are suitable for proteolytic cleavage; and
(c) measurement of the proportion of split fusion protein by means of confocal fluorometric methods.

13. A method for analysis of intracellular protease activity, comprising the steps:
(a) insertion of the nucleic acid sequence according to claim 7 and/or the vector according to claim 8 into the cell to be tested so that the autofluorescent fusion protein as defined in claims 1 to 6 is expressed intracellularly;
(b) incubation under conditions which are suitable for an expression and proteolytic cleavage of the fusion protein; and
(c) determination of the protease activity occurring intracellularly by means of confocal fluorometric methods.

14. A method for analysis of intracellular protease inhibiting activity, comprising the steps:
(a) insertion of the nucleic acid sequence according to claim 7 and/or the vector according to claim 8 into the cell to be tested so that the autofluorescent fusion protein as defined in claims 1 to 6 is expressed intracellularly;
(b) incubation under conditions which are suitable for an expression and proteolytic cleavage of the fusion protein; and
(c) determination of the protease inhibiting activity occurring intracellularly by means of confocal fluorometric methods.

15. A method according to one of the claims 11 to 14, whereby a dual-colour confocal fluorometric measurement method is used as measurement method.

16. A method according to claim 15, wherein the dual-colour fluorescence cross-correlation spectroscopy (KK-FCS), the confocal fluorescence coincidence analysis (CFCA) or the 2D fluorescence intensity distribution analysis (2D-FIDA) is used as the measurement method.

17. A method according to claim 15, wherein the proportion of molecules displaying a fluorescence energy transfer is determined in addition to the dual-colour confocal fluorometric measurement methods.

18. Use of a method according to one of the claims 11 to 17 for the screening-based, evolutive optimization of biomolecules with proteolytic activity and/or the generation of biomolecules with proteolytic activity by screening-based directed evolution.


SEQUENCE LISTING (12 sequences)

SEQ ID NO: 1 - protein, 487 aa, Artificial sequence
  Autofluorescent fusion protein derived from GFP variants of e.g. Aequorea victoria and/or Discoma sp.
MTMITPSLHAMASKGEELFTGVVPILVELDGDVNGHKFSVSGEGEGDATYGKLTLKFICTTGKLPVPWPTLVTTLCYGVQCFSRYPDHMKRHDFFKSAMPEGYVQERTIFFKDDGNYKTRAEVKFEGDTLVNRIELKGIDFKEDGNILGHKLEYNYNSHNVYIMADKQKNGIKVNFKTRHNIEDGSVQLADHYQQNTPIGDGPVLLPDNHYLSTQSALSKDPNEKRDHMVLLEFVTAAGITHGMDELYNQSTLEDPRVPVATMRSSKNVIKEFMRFKVRMEGTVNGHEFEIEGEGEGRPYEGHNTVKLKVTKGGPLPFAWDILSPQFQYGSKVYVKHPADIPDYKKLSFPEGFKWERVMNFEDGGVVTVTQDSSLQDGCFIYKVKFIGVNFPSDGPVMQKKTMGWEASTERLYPRDGVLKGEIHKALKLKDGGHYLVEFKSIYMAKKPVQLPGYYYVDSKLDITSHNEDYTIVEQYERTEGRHHLFL

SEQ ID NO: 2 - protein, 506 aa, Artificial sequence
  Autofluorescent fusion protein derived from GFP variants of e.g. Aequorea victoria and/or Discoma sp.
MTMITPSLHAMASKGEELFTGVVPILVELDGDVNGHKFSVSGEGEGDATYGKLTLKFICTTGKLPVPWPTLVTTLCYGVQCFSRYPDHMKRHDFFKSAMPEGYVQERTIFFKDDGNYKTRAEVKFEGDTLVNRIELKGIDFKEDGNILGHKLEYNYNSHNVYIMADKQKNGIKVNFKTRHNIEDGSVQLADHYQQNTPIGDGPVLLPDNHYLSTQSALSKDPNEKRDHMVLLEFVTAAGITHGMDELYNQSTLEDPDIPTTENLYFQSGTVDADPRVPVATMRSSKNVIKEFMRFKVRMEGTVNGHEFEIEGEGEGRPYEGHNTVKLKVTKGGPLPFAWDILSPQFQYGSKVYVKHPADIPDYKKLSFPEGFKWERVMNFEDGGVVTVTQDSSLQDGCFIYKVKFIGVNFPSDGPVMQKKTMGWEASTERLYPRDGVLKGEIHKALKLKDGGHYLVEFKSIYMAKKPVQLPGYYYVDSKLDITSHNEDYTIVEQYERTEGRHHLFL

SEQ ID NO: 3 - protein, 547 aa, Artificial sequence
  Autofluorescent fusion protein derived from GFP variants of e.g. Aequorea victoria and/or Discoma sp.
MTMITPSLHAMASKGEELFTGVVPILVELDGDVNGHKFSVSGEGEGDATYGKLTLKFICTTGKLPVPWPTLVTTLCYGVQCFSRYPDHMKRHDFFKSAMPEGYVQERTIFFKDDGNYKTRAEVKFEGDTLVNRIELKGIDFKEDGNILGHKLEYNYNSHNVYIMADKQKNGIKVNFKTRHNIEDGSVQLADHYQQNTPIGDGPVLLPDNHYLSTQSALSKDPNEKRDHMVLLEFVTAAGITHGMDELYNQSTLEDPTSQHGTNSEIEEYLKVLYDYDIPTTENLYFQSGTVDAGADAGKKKDQKDDKVAEQASKDPRVPVATMRSSKNVIKEFMRFKVRMEGTVNGHEFEIEGEGEGRPYEGHNTVKLKVTKGGPLPFAWDILSPQFQYGSKVYVKHPADIPDYKKLSFPEGFKWERVMNFEDGGVVTVTQDSSLQDGCFIYKVKFIGVNFPSDGPVMQKKTMGWEASTERLYPRDGVLKGEIHKALKLKDGGHYLVEFKSIYMAKKPVQLPGYYYVDSKLDITSHNEDYTIVEQYERTEGRHHLFL

SEQ ID NO: 4 - protein, 32 aa, Artificial sequence
  Segment at cleavage site derived from internal fragment of GFP, e.g., Aequorea victoria and/or Discoma sp.
QSTLEDPDIPTTENLYFQSGTVDADPRVPVAT

SEQ ID NO: 5 - protein, 7 aa, Artificial sequence
  Proteolytic cleavage site derived from tobacco etch virus (TEV) protease
ENLYFQS

SEQ ID NO: 6 - protein, 4 aa, Artificial sequence
  Proteolytic cleavage site derived from mammalian Factor Xa
IEGR

SEQ ID NO: 7 - protein, 4 aa, Artificial sequence
  Proteolytic cleavage site derived from mammalian plasmin
KXYK

SEQ ID NO: 8 - protein, 9 aa, Artificial sequence
  Proteolytic cleavage site derived from mammalian thrombin
VGPRSFLLK

SEQ ID NO: 9 - protein, 7 aa, Artificial sequence
  Proteolytic cleavage site derived from mammalian angiotensin converting enzyme (ACE)
GKYAPWV

SEQ ID NO: 10 - protein, 8 aa, Artificial sequence
  Proteolytic cleavage site derived from HIV protease
SQNYPIVQ

SEQ ID NO: 11 - protein, 11 aa, Artificial sequence
  Proteolytic cleavage site derived from human cytomegalovirus (hCMV) protease
RGVVNASSRLA

SEQ ID NO: 12 - protein, 8 aa, Artificial sequence
  Proteolytic cleavage site derived from herpes simplex virus (HSV) protease
LVLASSSF